(19) United States
(12) Patent Application Publication
(10) Pub. No.: US 2026/0284433 A1
(43) Pub. Date: Sep. 24, 2026

(54) FUNCTION-GUIDED RADIATION THERAPY PLAN GENERATION METHOD, APPARATUS, ELECTRONIC DEVICE AND PROGRAM PRODUCT

(71) Applicant: THE HONG KONG POLYTECHNIC UNIVERSITY, Hong Kong (CN)

(72) Inventors: Jing CAI, Hong Kong (CN); Tianyu XIONG, Hong Kong (CN); Ge REN, Hong Kong (CN)

(21) Appl. No.: 19/409,891

(22) Filed: Dec. 5, 2025

(30) Foreign Application Priority Data
Mar. 18, 2025 (CN) 202510316395.4

(51) Int. Cl. A61N 5/10; G16H 20/40

(52) U.S. Cl. CPC A61N 5/1031; G16H 20/40

(57) ABSTRACT

Method, apparatus, electronic device and program product of function-guided radiation therapy (RT) plan generation useful to medical treatment for a target user are provided. In the method, anatomical data and functional data corresponding to the target user are acquired. The anatomical data at least models an anatomical structure of a tumor target volume and organ(s) at risk of the target user. The functional data models a distribution condition of a lung function of the target user. A target dose distribution is determined according to the anatomical data and the functional data. A function-guided RT plan for the target user is then generated based on the target dose distribution. Advantageously, using function-guided RT plan can reduce the chance of radiation pneumonia and improve the effect of RT.

### CROSS-REFERENCE TO RELATED APPLICATIONS

This application claims priority to China Invention Patent Application No. 202510316395.4 filed Mar. 18, 2025, the disclosure of which is incorporated by reference herein in its entirety.

### TECHNICAL FIELD

The present application belongs to the field of medical technology, and in particular relates to function-guided radiation therapy plan generation method, apparatus, electronic device, and program product.

### BACKGROUND

Lung cancer is one of the most common malignant tumors, and radiation therapy (RT) is one of the generally used means to treat tumors, and over 77% of patients with lung cancer need to receive RT during the treatment. Under the influence of factors such as radioactive penetration, sensitivity of organ tissues and cumulative effect of multiple treatments, RT may cause certain damage to other tissues in the lung and even other organs around while killing or preventing the growth of lung tumor cells in patients, resulting in a series of adverse reactions such as lung fibrosis or radiation pneumonitis.

In the prior art, the formulation of the traditional RT plan for the lung is generally only dependent on anatomical images such as computed tomography (CT) images of patients, so as to ensure that the tumors receive enough irradiation dose and to minimize the damage to the normal tissues. However, anatomical images can only provide information on the anatomical structure and cannot reflect the functional heterogeneous distribution of the lungs, making it impossible to distinguish the function of normal tissues when formulating RT plans, resulting in unnecessary high-dose irradiation to high-function regions, and affecting the treatment effect.

### SUMMARY

Embodiments of the present disclosure provide function-guided RT plan generation method, apparatus, electronic device, and program product, which can reduce the chance of radiation pneumonia and improve the effect of RT.

In a first aspect, certain embodiments of the present disclosure provide a function-guided RT plan generation method. The method includes: acquiring anatomical data and functional data corresponding to a target user, wherein the anatomical data at least models an anatomical structure of a tumor target volume and organ(s) at risk of the target user, and wherein the functional data is used to model a distribution of the lung function of the target user; determining a target dose distribution according to the anatomical data and the functional data; and determining a function-guided RT plan for the target user based on the target dose distribution.

In a second aspect, certain embodiments of the present disclosure provide a function-guided RT plan generation apparatus. The apparatus includes: a data acquisition module for acquiring anatomical data and functional data corresponding to a target user, wherein the anatomical data at least models an anatomical structure of a tumor target volume and organ(s) at risk of the target user, and wherein the functional data is used to model a distribution condition of a lung function of the target user; a dose distribution prediction module for determining a target dose distribution according to the anatomical data and the functional data; and an automatic RT plan generation module for determining a function-guided RT plan for the target user based on the target dose distribution.

In a third aspect, certain embodiments of the present disclosure provide an electronic device including a memory, a processor, and a computer program stored in the memory and runnable on the processor, wherein the processor executing the computer program implements the steps of the function-guided RT plan generation method mentioned above in the first aspect.

In a fourth aspect, certain embodiments of the present disclosure provide a computer readable storage medium have stored thereon a computer program that, when executed by a processor, implements the steps of the function-guided RT plan generation method in the first aspect mentioned above.

In a fifth aspect, certain embodiments of the present disclosure provide a computer program product that, when run on an electronic device, causes the electronic device to perform the function-guided RT plan generation method in the first aspect mentioned above.

Compared with the prior art, the embodiments of the present disclosure have the following beneficial effects.

In the present disclosure, the target dose distribution is jointly determined according to the anatomical data and the functional data. Compared with a RT plan that depends on anatomical images only, the heterogeneous distribution information of the lung function provided by the lung function images can be additionally integrated. As a result, when the target dose distribution is determined, a high-function region can be protected as much as possible, the lung function damage caused by RT is favorably reduced, and the effect of RT is improved. Moreover, the target dose distribution is automatically determined and the function-guided RT plan is automatically generated based on the anatomical data and functional data, eliminating a need for manual analysis and planning to formulate the RT plan. It can effectively improve the efficiency, consistency and quality of the function-guided RT plan.

### BRIEF DESCRIPTION OF DRAWINGS

FIG. 1 is a flowchart of a function-guided RT plan generation method provided by certain embodiments of the present disclosure.

FIG. 2 is a schematic diagram of a function-guided dose prediction model provided by certain embodiments of the present disclosure.

FIG. 3 is a schematic diagram of another function-guided dose prediction model provided by certain embodiments of the present disclosure.

FIG. 4 is a schematic diagram of a function-guided RT plan generation apparatus provided by certain embodiments of the present disclosure.

FIG. 5 is a schematic diagram of an electronic device provided by certain embodiments of the present disclosure.

### DETAILED DESCRIPTION

In the following description, for purposes of explanation and not limitation, specific detail are set forth, such as specific system structures, techniques, etc., in order to provide a thorough understanding of embodiments of the present disclosure. It will be apparent, however, to those skilled in the art that the present disclosure may be practiced in other embodiments without these specific details. In other instances, detailed descriptions of well-known systems, apparatuses, circuits, and methods are omitted so as not to obscure the description of the present disclosure with unnecessary details.

It should be understood that when used in the present specification and the appended claims, the term “include” indicates the presence of described features, integers, steps, operations, elements and/or assemblies, but does not preclude the presence or addition of one or more other features, integers, steps, operations, elements, assemblies and/or collections thereof.

It should also be understood that the term “and/or” as used in the specification of this application and the appended claims refers to any and all possible combinations of one or more of the associated listed items, and includes these combinations.

In addition, in the description of the present disclosure specification and the appended claims, the terms “first”, “second”, “third”, etc. are only used to distinguish the descriptions and cannot be understood as indicating or implying relative importance.

Reference to “one embodiment” or “some embodiment”, etc. in the specification of the present disclosure mean that a specific feature, structure, or characteristic described in connection with the embodiment is included in one or more embodiments of the present disclosure. Thus, phrases such as “in one embodiment,” “in some embodiments,” “in some other embodiments,” “in yet other embodiments”, etc., appearing in various places in this specification are not necessarily all referring to the same embodiment, but rather mean “one or more but not all embodiments,” unless specifically emphasized otherwise.

### Embodiments 1

The establishment of treatment plan is a key step in the implementation of RT, and its core is to establish a plan that can guide the RT machine to perform RT and deliver the expected dose distribution. A conventional RT plan aims to deliver sufficient and uniform dose to tumor while minimizing dose to normal tissue especially organs at risk. Therefore, at present, when formulating a RT plan, tumor target volume and normal tissue are mainly contoured based on anatomical images, and the parameters in the RT plan are manually and repeatedly adjusted to ensure that the tumor target volume can obtain a sufficient and uniform dose while minimizing dose to normal tissue. The RT plan formulation process is time-consuming and laborious. It is affected by factors such as clinical experience and subjectivity of the planners. The formulated RT plan may have problems of inconsistency and low quality.

In view of this, certain embodiments of the present disclosure provide a function-guided RT plan generation method to automate the formulation process of the function-guided RT plan. It first acquires anatomical data and functional data corresponding to the target user. The anatomical data at least models an anatomical structure of a tumor area and one or more organs at risk of the target user. The functional data is used to model a distribution of a lung function of the target user. Then, a target dose distribution is determined according to the anatomical data and the functional data. A function-guided RT plan for the target user is generated based on the target dose distribution.

The function-guided RT plan generation method provided by the aforesaid certain embodiments of the present disclosure incorporates the lung function information into RT plan generation and preferentially protects high-function regions. By combining the anatomical data including the tumor region and the organ(s) at risk distribution, and the functional data including the lung function distribution, it can predict target dose distribution where the dose can be redistributed from the high-function lung region to the low-function lung region, while meeting the traditional dose distribution constraints. Furthermore, the function-guided RT plan is generated according to the target dose distribution, so that the lung function of a target user is better protected while other conventional planning objectives including tumor coverage and the protection of organ(s) at risk are ensured. Consequently, radiation-induced lung injury is effectively reduced, and the effect of RT is improved. The function-guided RT plan is not affected by artificial factors, and the consistency, efficiency and quality of the produced plans are effectively ensured.

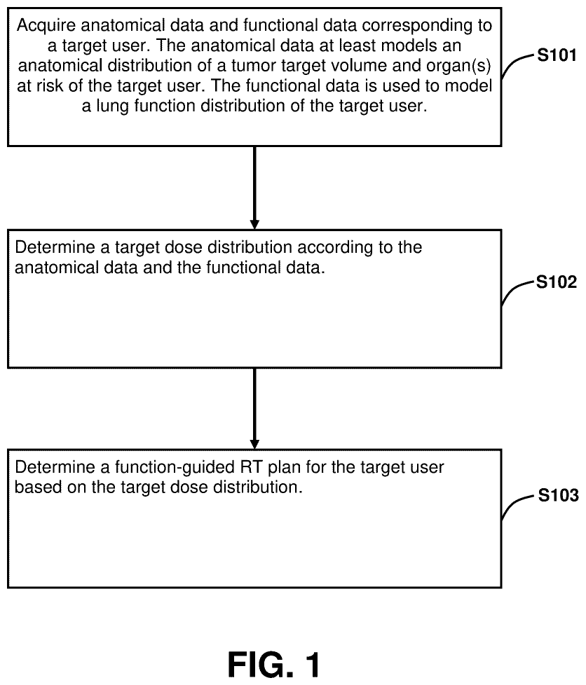

FIG. 1 shows a flowchart of a function-guided RT plan generation method provided by the aforesaid certain embodiments of the present disclosure. The generation method is detailed as follows.

S101: Acquire anatomical data and functional data corresponding to a target user. The anatomical data at least models the anatomical structure of the tumor area and the organ(s) at risk of the target user. The functional data is used to model the lung function distribution of the target user.

It should be understood that the target user generally refers to a patient who is to undergo RT.

It should be understood that the anatomical data at least models the anatomical structure of the tumor target volume (also called Planning Target Volume (PTV)) of the organ to be treated of the target user and the organ(s) at risk. The aforesaid certain embodiments of the present disclosure are illustrated by taking the lung as an example of the organ to be treated. Therefore, the anatomical data at least models the anatomical structure of the lung tumor target volume and the organ(s) at risk. An individual organ at risk generally refers to the normal tissue or organ that may be involved in the range of the radiation damage, that is, the organ or tissue to be protected around the tumor target volume.

In some embodiments, the anatomical data may further model anatomical structures of other organs or tissues associated with the organ to be treated.

It should be understood that the anatomical structures may include contours, sizes and locations of organs or tissues, etc. Optionally, the anatomical data may be represented as one or more of an image, a text, a three-dimensional model, etc., which may be specifically set according to actual application requirements.

It should be understood that the functional data at least models the functional distribution of the organ to be protected of the target user. The functional data is used to model the distribution of the lung function of the target user, such as the distribution of the ventilation function, etc. It should be understood that the functional data may take the form of one or more of an image, a text, graphic and text data, etc., which may be set in accordance with actual application requirements.

As one example, the functional data may be in the form of images. That is, the functional data may be functional images including, but not limited to, single-photon emission computed tomography (SPECT) ventilation images, SPECT perfusion images, magnetic resonance imaging (MRI) ventilation images, MRI perfusion images, positron emission tomography (PET) ventilation images and PET perfusion images.

In some embodiments, the anatomical data may include CT images, planning target volume contour maps, and contour maps of organs at risk, the functional data may include lung function images (i.e. images that directly model the lung function) and lung isofunctional contour maps (i.e. structured images obtained based on lung function images).

Optionally, after acquiring the CT image and the lung function image, the lung function image can be pre-processed by registration and resampling so that the pre-processed lung function image can be aligned with the location and resolution of the CT image. As a result, the lung function information can be provided more intuitively and accurately later.

It should be pointed out that the embodiments of the present disclosure are illustrated using the lung RT scenario as an example. When the above-mentioned RT plan generation method is applied to other organ RT scenarios, the anatomical data is at least used to model the anatomical structure of the tumor target volume corresponding to the organ to be treated and its organ(s) at risk (generally referred to the organ close to the tumor), and the functional data can be used to reflect the functional distribution of the organ to be protected. The organ to be protected can be any organ at risk close to the tumor. For example, when the organ to be protected is liver, the functional data can be used to reflect the spatial heterogeneity distribution information of liver function, etc.

S102: Determine a target dose distribution according to the anatomical data and the functional data.

A dose distribution generally refers to the spatial distribution of radiation dose in the patient's body structure (also known as tissue, such as the lungs), which can generally be described as the radiation energy per unit mass.

Radiation may damage the patient's lung tissue, potentially lead to radiation-induced lung injury, and affect the patient's lung function. In order to reduce the chance of radiation-induced lung injury, when formulating a RT plan for the target patient, a comprehensive analysis can be performed based on anatomical data and functional data to obtain a clinically preferable target dose distribution which can preferentially protect high-function lung regions.

It should be understood that in determining the target dose distribution in combination with information of at least two different modalities including anatomical data that models the tumor target region and functional data that models the lung function distribution, automatic analysis can be performed in combination with RT planning objectives of damaging tumor cells and protecting organs at risk and high-function lung regions. That is, in determining the target dose distribution, the current lung function distribution of the patient is fully considered, and the high-function lung region is avoided as much as possible. The radiation dose is concentrated to the tumor region (i.e. the tumor target volume region) and the low-function region, so that the damage to the lung function of the patient during the subsequent RT based on the target dose distribution can be minimized, and further, the chance of causing radiation-induced lung injury to the patient can be reduced.

S103: Determine a function-guided RT plan for the above-mentioned target user based on the above-mentioned target dose distribution.

It should be understood that a RT plan may be used to guide a RT machine to perform RT. For example, the RT plan may be presented as a set of executable machine parameters of the RT machine such as position, motion trajectory, motion speed, dose rate (which may further be expressed as beamlet intensity), and sub-field weights, etc. of a multileaf collimator (MLC).

In certain embodiments of the present disclosure, since the anatomical data can model the anatomical structure of the tumor target volume and the organ(s) at risk (that is, the distribution of the tumor target volume and the surrounding organ tissues to be protected) of the target user, and the functional data can model the distribution of the lung function of the target user, it follows that the distributions of the tumor target volume, the organ(s) at risk and the high-low distribution of the lung function of the user can be fully known according to the data. When the target dose distribution is determined, the tissue with good lung function can be avoided as much as possible. That is, the high-function region is avoided, and the dose distribution is concentrated on the tumor and the low-function region. As a result, the radiation dose to the high-function tissue and the damage to the lung function can be reduced to the greatest extent, and the target dose distribution beneficial to the protection of the lung function is obtained. Furthermore, the function-guided RT plan corresponding to the target user is automatically generated according to the target dose distribution. The formulation efficiency, the consistency and the quality of the function-guided RT plan can be effectively improved while the chance of radiation-induced lung injury to the target user is reduced and the effect of RT is improved.

In some embodiments, the step S102 includes: using a trained prediction model to obtain the target dose distribution, wherein the prediction model is designed to establish a relationship between the anatomical and functional data and corresponding dose distribution, thereby obtaining the target dose distribution according to the anatomical and functional data of the target user.

It should be understood that the prediction model may be a machine learning model constructed based on a network structure such as a convolutional neural network, a U-Net, a generative adversarial network (GAN), a graph neural network (GNN), or a transformer. The present disclosure is not specifically limited to any particular network structure in implementing the prediction model.

As one example, the trained prediction model may be a model obtained by fine-tuning a general large model using constructed training samples (generally including functional data, anatomical data corresponding to the user, and actual function-guided planning dose distribution). As the large model is a deep learning model with a massive number of parameters (generally billions or even hundreds of billions), a training ssubset is adopted to fine-tune the general large model which is trained by a large amount of data, and the large model can learn the relationship between the anatomical structure and the lung function and the corresponding function-guided planning dose distributions for different patients. The fine-tuned large model can better predict the function-guided dose distribution matched with the anatomical distribution and the lung function distribution according to the anatomical data and the functional data, and the accuracy of the predicted target dose distribution is improved.

In certain embodiments of the present disclosure, since the model can better learn the complex relationship between the anatomical and functional data and the corresponding function-guided planning dose distribution, the trained prediction model can more accurately predict the dose distribution matched with the anatomical and function distribution of a target user according to the anatomical data and the functional data, improves the accuracy of the obtained target dose distribution.

In some embodiments, the prediction model includes an anatomical encoding network, a function encoding network, a fusion network, and a decoding network. The process of using the trained prediction model to obtain the target dose distribution includes:
encoding the anatomical data through the anatomical encoding network to obtain anatomical features, and encoding the functional data through the functional encoding network to obtain functional features;
fusing the anatomical features and the functional features through the fusion network to obtain fusion features; and
predicting the dose distribution matched with the anatomical and function distribution according to the fusion features through the decoding network to obtain the target dose distribution.

Specifically, considering that the anatomical data and the functional data are data reflecting different modal information, in order to improve the feature accuracy, the anatomical data and the functional data can be respectively encoded (also known as feature extraction processing) by different encoders to obtain anatomical features corresponding to the anatomical data and functional features corresponding to the functional data. Since the anatomical encoding network and the functional encoding network can focus on feature extraction of the corresponding modal data, the unique information of each modal data can be well extracted and retained. As a result, it is conducive to providing richer feature representation for subsequent feature fusion and prediction of target dose distribution, and the prediction model can be well adapted to changes of different modal data. The generalization ability of the prediction model on data corresponding to different patients is improved.

Optionally, the fusion network may fuse the anatomical features with the functional features based on attention mechanisms (such as channel attention or spatial attention, etc.). Since the attention mechanism can enable the fusion network to pay more attention to the characteristics that are helpful to the dose prediction task in the fusion process, the fusion network based on the attention mechanism can improve the accuracy of the characteristic representation and obtain the fusion characteristics with a higher accuracy. It is conducive to the subsequent guiding decoding network to more accurately predict the function-guided planning dose distribution and improving the accuracy of the target dose distribution prediction.

In some embodiments, the fusion network may fuse anatomical features with functional features based on channel attention and spatial attention. Since channel attention may focus on the channel dimension of the feature, it highlights the channels associated with the dose prediction task. Since spatial attention may focus on the spatial dimension of the feature, it highlights the regions that contribute the most to the dose prediction task. Thus, anatomical and functional features are fused together based on channel attention and spatial attention. The feature expression can be enhanced from different dimensions, and complementary feature information can be extracted from anatomical features and functional features of different modalities for fusion. It follows that that the obtained fusion features are further enhanced. The prediction model can thus better learn and understand the complex relationship between the anatomical and functional features and the corresponding function-guided planning dose distribution.

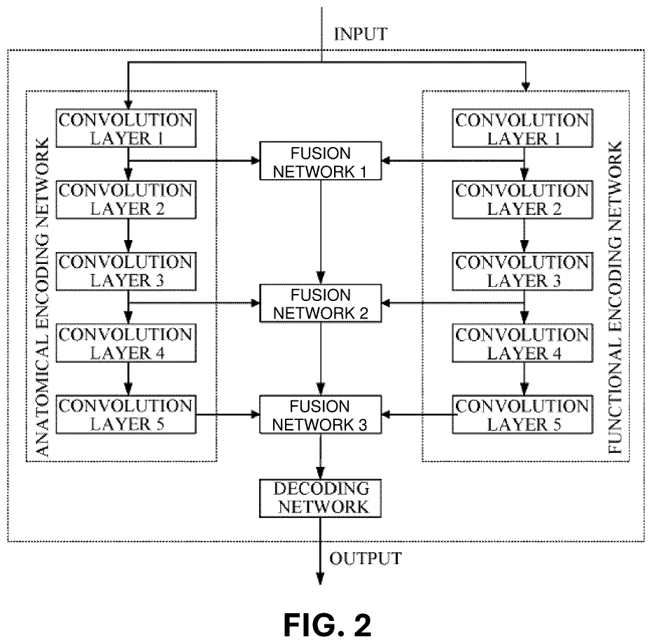

FIG. 2 shows a schematic diagram of the prediction model in accordance with some embodiments.

Refer to FIG. 2. The prediction model may include n fusion networks (n is greater than 1, for example, the prediction model in FIG. 2 includes 3 fusion networks). The anatomical encoding network and the functional encoding network may include m convolution layers (m is greater than 1, m is generally greater than or equal to n, as the prediction model in FIG. 2 includes 3 convolution layers). Different fusion networks are used to fuse the anatomical and functional features extracted from different convolution layers.

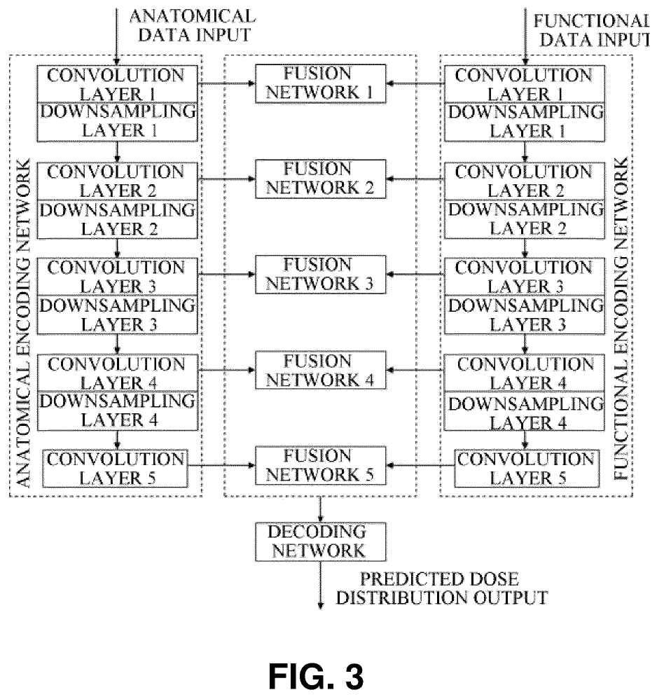

FIG. 3 shows a schematic diagram of the prediction model in some other embodiments.

Refer to FIG. 3. The prediction model may include n fusion networks (n is greater than 1, for example, the prediction model in FIG. 3 includes 5 fusion networks), the anatomical encoding network and the functional encoding network may include m convolution layers (m is greater than 1, for example, the prediction model in FIG. 3 includes 5 convolution layers) and m−1 downsampling layers. Different fusion networks are used to fuse anatomical features and functional features extracted by different convolution layers at different scales. That is, through the above-mentioned arrangement, the characteristics of different scales obtained by each convolution layer of the anatomical encoding network and the functional encoding network as well as the fusion characteristics of different scales obtained by fusion of different fusion networks can be taken as inputs of the decoding network, so that the decoding network can perform final dose distribution prediction according to each characteristic of different scales.

Since different convolution layers in that anatomical encoding network and the functional code network can extract features of different scales, the anatomical features and the functional feature extracted by different convolution layers are fused through different fusion network. As a result, multi-scale information can be better captured. The feature representation is further enhanced, which is conducive to improving the overall performance of the prediction model.

In certain embodiments of the present disclosure, data of different modalities are encoded and processed through different encoding networks. It follows that the encoding network can focus on extracting feature information of a single modality, thereby improving the accuracy of the obtained features. Furthermore, the anatomical features and functional features of different modalities are fused, which can better utilize the complementarity between anatomical features and functional features and integrate to obtain multimodal fusion features. Furthermore, based on the fusion feature, the dose distribution matched with the anatomical structure distribution and the lung function distribution can be predicted more accurately. The prediction accuracy, robustness and other performances of the prediction model can be better enhanced. Meanwhile, the clinical availability, accuracy and the like of predicting the dose distribution can be effectively enhanced.

In some embodiments, the step S102 further includes a step of training the prediction model. The step of training the prediction model includes the following steps:
constructing a plurality of training samples, wherein an individual training sample includes anatomical data and functional data corresponding to a user;
taking the individual training sample as inputs of the prediction model to be trained to obtain the predicted dose distribution;
determining a loss value according to the predicted dose distribution corresponding to the individual training sample and an actual function-guided planning dose distribution; and
updating parameters of the prediction model to be trained according to respective loss values computed for the plurality of training samples to obtain the trained prediction model mentioned above.

It should be understood that the actual function-guided plan dose distribution corresponding to the individual training sample generally refers to the label value of the individual training sample, and the actual function-guided plan dose distribution can be manually formulated by an experienced clinician or automatically generated by an intelligent algorithm such as a large model, or can be specifically set according to actual application requirements.

It should be understood that when constructing the plurality of training samples, a certain number (such as 1000) of respective training samples are generally constructed to ensure the accuracy and generalizability of the trained prediction model. Optionally, after the plurality of training samples is constructed, a training sample set, a validation sample set, and a test sample set may be formed according to the constructed training samples. The training sample set is used to train the prediction model to be trained. The validation sample set can be used for evaluating the performance of the prediction model in a training process so as to help select hyperparameters and avoid over-fitting boarding. The test sample set can be used to evaluate the performance of the resultant trained model (such as generalization ability, etc.). By reasonably dividing the training sample set, the validation sample set and the test sample set, the prediction model can be effectively evaluated and optimized, and the prediction performance of the prediction model on new data is ensured.

Optionally, when updating the parameters of the prediction model to be trained according to the respective loss values, the parameters of the prediction model may be updated by one or more optimization algorithms, such as a gradient descent algorithm, a momentum or an adaptive learning rate, etc., which may be specifically set according to an actual application scenario.

In some embodiments, after the parameters of the above-mentioned prediction model to be trained are updated according to the above-mentioned respective loss values, in case that the updated prediction model does not meet the preset training requirements, the step of training the prediction model may be repeatedly performed based on the updated prediction model until the latest updated prediction model meets the training requirements to obtain the trained prediction model. The training requirements may include one or more of iteration number requirements (such as the iteration number being greater than a number threshold, such as 100) and a performance requirement (such as the prediction error being less than a set error threshold, such as 0.1).

In certain embodiments of the present disclosure, through training the prediction model, the prediction model learns the complex relationship among the anatomical data, the functional data and the function-guided dose distribution. It thereby learns to predict the influence of the distribution conditions of the anatomical structure of the tumor target volume, organ(s) at risk, and the lung function on the actual function-guided planning dose distribution. The correlation between the lung function and the dose distribution can be fully considered when the function-guided dose distribution is predicted, so that the prediction accuracy of the prediction model is guaranteed.

In some embodiments, the determining of the loss value according to the predicted dose distribution corresponding to the individual training sample and the actual function-guided planning dose distribution includes: determining the loss value corresponding to the individual training sample according to the predicted dose distribution, the actual function-guided plan dose distribution, and a functional weight of a target voxel, wherein the functional weight is determined according to the functional data in the individual training sample.

Voxel, also known as volume pixel, refers to the smallest unit in three-dimensional space and is generally used to describe density, signal intensity or other physical quantities in medical images. In RT, voxels are generally used to discretize the dose distribution. That is, the dose distribution is generally calculated in units of voxel, and the dose value of each voxel generally represents a radiation dose received at that location.

Optionally, the target voxel may be a voxel determined based on the tumor target volume. For example, the target voxel may be a tumor target volume and a voxel corresponding to the organ at risk. Alternatively, the target voxel may be a voxel determined based on the lung function distribution reflected by the functional data. For example, the target voxel may be a voxel corresponding to a high-function region. Yet alternatively, the target voxel may be a voxel determined based on the tumor target volume and the functional distribution, and may be specifically set according to actual application requirements.

When calculating the loss value corresponding to a training sample, the prediction error value between the predicted dose value and the actual dose value of the target voxel can be calculated first according to the predicted dose distribution and the actual function-guided planning dose distribution. For the prediction error value corresponding to the target voxel, the prediction error value can be weighted according to the functional weight of the target voxel, and the weighted prediction error value can be used as the loss value corresponding to the target voxel. For the non-target voxel (if there is a non-target voxel), the calculated prediction error value can be directly used as the loss value corresponding to the voxel. Further, the loss value corresponding to the training sample is calculated according to the loss value corresponding to each individual pixel.

Since the functional weight of the target voxel is determined based on the lung function distribution, the dose difference value of the target voxel can be enlarged or reduced in combination with the actual lung function corresponding to the target voxel through the above-mentioned processing, so that the prediction accuracy of a high-function region can be improved. Furthermore, the high-function tissue can be better protected while the treatment of tumor tissue is well realized. Effectiveness and safety of RT is improved. That is, the effect of RT is improved.

Optionally, a preset loss function for calculating the loss value may include an error loss function and a dose-volume histogram (DVH)-based loss function.

As one example, the error loss function may include a mean absolute error (MAE) loss function, and the dose volume-based loss function may include a DVH loss function. The MAE loss function is generally used to measure the difference between the predicted dose value and the actual dose value to obtain an error loss value, and the predicted dose value of each target voxel is made as close as possible to the actual dose value through the voxel-level error loss. The loss function of DVH is generally used to measure the difference between the predicted DVH and the actual DVH to obtain the error loss value of the DVH. Through the loss of the DVH so as to make the clinically important DVH metrics of the predicted dose distribution are close to the actual DVH metrics, the overall dose distribution characteristic is optimized. The accuracy of the predicted dose distribution is effectively improved.

As one example, the MAE loss function can be expressed as the following form:

$${L}_{MAE} = \frac{1}{{n}_{total}} {\sum }_{i ∈ {n}_{total}} \vert {D}_{i}^{pred} - {D}_{i}^{plan} \vert + \frac{1}{{n}_{lung}} {\sum }_{j ∈ {n}_{lung}} {f}_{j} \vert {D}_{j}^{pred} - {D}_{j}^{plan} \vert$$

where: L_{MAE} represents the error loss value; nₜₒₜₐₗ represents the total number of all voxels; n_{lung} represents the number of target voxels (in illustrating the present disclosure, the target voxel is a voxel corresponding to a lung structure, also known as a lung voxel);

$${D}_{i}^{pred}$$

represents the predicted dose value of the i-th voxel;

$${D}_{i}^{plan}$$

represents the actual dose value of the i-th voxel; fⱼ represents the functional weight of the j-th lung voxel;

$${D}_{j}^{pred}$$

represents the predicted dose value of the j-th lung voxel;

$${D}_{j}^{plan}$$

represents the actual dose value of the j-th lung voxel; ∥ means taking the absolute value; and Σ means the accumulation operator.

It should be understood that in certain embodiments, the target voxel may further include voxels corresponding to organ tissues other than the lungs.

As one example, the DVH loss function may calculate a dose volume parameter loss value based on the DVH. Optionally, the DVH may be approximated by a series of moments of different orders, and the dose volume loss value is obtained according to the error of the predicted moment and the actual moment.

As one example, the DVH loss function can be expressed as the following form:

$${L}_{DVH} = {\sum }_{p ∈ P , s ∈ S} {‖ {M}_{p}^{{s}^{pred}} - {M}_{p}^{{s}^{plan}} ‖}_{2}^{2}$$

where: L_{DVH} represents the DVH loss value; p represents the order; s represents the s-th structure (i.e. organ, such as lung, heart, and tumor target volume, etc.) in the tissue and organ set S involved in the RT plan design; ∥ ∥₂ represents the L2 norm (also called the second norm);

$${M}_{p}^{{s}^{pred}}$$

represents the predicted moment of the s-th organ; and

$${M}_{p}^{{s}^{plan}}$$

represents the actual moment of the s-th organ.

It should be understood that the structure contained in the organ tissue set S involved in the RT plan design may be manually set or automatically generated based on empirical data. The present disclosure does not impose any specific limitation on this aspect.

Optionally, the moments can be expressed as the following form:

$${M}_{p}^{s} = {( \frac{1}{{n}_{s}} {\sum }_{i ∈ {n}_{s}} {D}_{i}^{p} )}^{\frac{1}{p}}$$

where:

$${M}_{p}^{s}$$

represents the moment of order p of the s-th structure; nₛ represents the number of voxels in the s-th structure; Dᵢ represents the dose value of the i-th voxel in the s-th structure; and p represents the order.

Optionally, for parallel organ(s) to be protected (including esophagus, heart, lung, etc.), p=(1,2) can be set to approximate the average dose. For serial organ(s) to be protected (such as spinal cord), p=(5,10) could be set to approximate the maximum dose. Furthermore, p=(2, 4, 6) may be set for expressing a PTV.

In some embodiments, the dose volume-based loss function may further include a dose-function histogram (DFH) loss function. Optionally, the DFH loss function can be expressed as the following form:

$${L}_{DFH} = {\sum }_{p ∈ P} {‖ {F}_{p}^{{L}^{pred}} - {F}_{p}^{{L}^{plan}} ‖}_{2}^{2}$$

where: L_{DFH} represents the dose function histogram loss value;

$${F}_{p}^{{L}^{pred}}$$

represents the predicted moment based on lung function distribution (equivalent to the predicted dose function histogram); and

$${F}_{p}^{{L}^{plan}}$$

represents the actual moment based on lung function distribution (equivalent to the actual dose function histogram).

Optionally, the moment based on the lung function (i.e. the dose function histogram) can be expressed as the following form:

$${F}_{p}^{L} = {( \frac{1}{{n}_{lung}} {\sum }_{i ∈ {n}_{lung}} {f}_{i} {D}_{i}^{p} )}^{\frac{1}{p}}$$

where:

$${F}_{p}^{L}$$

represents the moment based on the lung function (i.e. the dose function histogram); n_{lung} represents the number of lung voxels; and fᵢ represents the functional weight of the i-th lung voxel.

Optionally, the loss value corresponding to the finally calculated training sample can be expressed as the following form:

$${L}_{total} = {L}_{MAE} + {w}_{DVH} ( {L}_{DVH} + {L}_{DFH} )$$

where: Lₜₒₜₐₗ represents the total loss value corresponding to the training sample; L_{MAE} represents the MAE loss value; L_{DVH} represents the DVH loss value; L_{DFH} represents the dose function histogram loss value; and w_{DVH} represents the relative weight of the dose function histogram and the DVH. Optionally, w_{DVH} may take a value of 0.01.

In some embodiments, the step S103 includes: generating the function-guided RT plan according to the target dose distribution by a function-guided dose mimicking algorithm adapted to convert the target dose distribution into a RT plan in accordance with an optimization function including at least a target term based on the lung function.

It should be understood that in order to ensure the quality of the generated function-guided RT plan, the above-mentioned optimization function (also known as an objective function) can be used to guide the generation of a function-guided RT plan that meets the predicted target dose distribution clinical requirements and physical limitations of the RT machine, and that the generation process of the function-guided RT plan is realized with the optimization goal of minimizing the difference between the calculated dose distribution and the target dose distribution. That is, the preset optimization function may be determined based on this optimization problem.

In some embodiments, the optimization objective and constraints of this optimization problem (i.e. the above-mentioned optimization function) are expressed as:

$${min}_{x} \{ \begin{matrix}\frac{{w}_{PTV}}{{n}_{PTV}} {\sum }_{i ∈ PTV} {( {D}_{i}^{cal} - {D}_{i}^{pre} )}^{2} + \\ {\sum }_{s ∈ S} \frac{{w}_{s}}{{n}_{s}} {\sum }_{i ∈ s} H ( {D}_{i}^{cal} - {D}_{i}^{pre} ) {( {D}_{i}^{cal} - {D}_{i}^{pre} )}^{2} + \\ \frac{{w}_{F}}{{n}_{lung}} {\sum }_{i ∈ lung} H ( {D}_{i}^{cal} - {D}_{i}^{pre} ) {{f}_{i} ( {D}_{i}^{cal} - {D}_{i}^{pre} )}^{2}\end{matrix} \}$$

$$s . t .$$

$$x \geq 0$$

$$\vec{{D}^{cal}} = D \vec{x}$$

where:
x represents the fluence map, and the physical limitation of the RT machine requires that x≥0;

$${min}_{x}$$

represents the optimization goal of minimizing the difference between the planning dose distribution and the predicted dose distribution by optimizing the fluence map;
w_{PTV} represents the weight of the tumor target volume;
n_{PTV} represents the number of voxels in the tumor target volume;
wₛ represents the weight of the s-th structure in the organ tissue set S;
nₛ represents the number of voxels of the s-th structure in the organ tissue set S;

n_{lung} represents the number of target voxels (the target voxels in the embodiment of the present disclosure are voxels corresponding to lung structures);
w_{F} represents the weight of the structures corresponding to the target voxels (which can be used to reflect the clinical priority of the structures);

$${D}_{i}^{cal}$$

represents the calculated dose value of the i-th voxel (determined according to the fluence map);

$${D}_{i}^{pre}$$

represents the predicted dose value of the i-th voxel (determined according to the predicted dose distribution);
fᵢ represents the functional weight of the i-th voxel; and
D represents the dose matrix, generally used to describe the dose contribution of each beamlet to each voxel.

It should be understood that the functional weight of a non-target voxel (i.e. a voxel other than the target voxel) may be set to 1. If the target voxel includes a voxel corresponding to a lung structure, the functional weight of the lung voxel can be determined according to the lung function distribution condition reflected by the functional data, and the functional weight of the non-lung voxel can be regarded as 1.

Optionally, the weights of the structures of the planning target volume (i.e. tumor target volume), lung, spinal cord, esophagus, heart, and normal tissue are 800, 800, 600, 400, 400, and 200, respectively.

In order to improve the actual treatment effect, after the target dose distribution is determined, the determined target dose distribution can be used as optimization guidance. An optimization function corresponding to the optimization problem can be solved to generate a function-guided RT plan that meets the predicted target dose distribution, clinical requirements and physical limits of RT machines. Since the optimization function includes at least a target term based on the lung function, that is, the guided dose is transferred from the high-function region to the low-function region based on the actual lung function distribution corresponding to the target user, the high-function region can be preferentially protected when the target dose distribution is converted into the function-guided RT plan based on the function-guided dose mimicking algorithm. This process can automatically generate the function-guided RT plan based on the actual distribution of lung function, improve the formulation efficiency, consistency and quality of the function-guided RT plan, and is conducive to improving the effect of subsequent RT.

In some embodiments, the generating of the function-guided RT plan according to the target dose distribution by the function-guided dose mimicking algorithm includes:
solving the optimization function based on the target dose distribution to obtain a target fluence map; and
converting the target fluence map into machine parameters of a RT machine based on a MLC leaf sequence optimization algorithm and a direct aperture optimization algorithm to obtain the function-guided RT plan.

The MLC leaf sequence optimization algorithm is generally used to convert the fluence map into the specific leaf position and motion sequence of the MLC. The leaf position and motion sequence of the MLC determines the shape and intensity distribution of the beamlet, thereby determining the actual dose distribution.

Optionally, the multi-leaf grating sequence optimization algorithm includes, but is not limited to, a sequence optimization algorithm based on static segmentation.

Direct aperture optimization algorithms are generally used to directly optimize the leaf position and motion sequence of the MLC to achieve the desired dose distribution. Optionally, the direct aperture optimization algorithm includes, but is not limited to, an optimization algorithm such as Interior Point OPTimize (IPOPT) or a genetic algorithm.

In the present disclosure, the optimization function can be a function based on the fluence map. When generating a RT plan according to the predicted target dose distribution by a function-guided dose mimicking algorithm, the optimization function corresponding to the optimization problem can be first solved to obtain the optimal fluence map. After determining the optimal target fluence map, the machine parameters that can be used to guide the implementation of RT can be determined by the MLC leaf sequence optimization algorithm (which is assumed to be the initial machine parameters), so that the fluence map that is matched with the calculated optimal fluence map can be generated based on the machine parameters during the actual treatment. It is conducive to improving the effect of RT. In order to further improve the effect of RT, after the initial machine parameters are determined by the MLC leaf sequence optimization algorithm, the obtained initial machine parameters can be further optimized by the direct aperture optimization algorithm to obtain the final machine parameters.

It should be understood that the size of the sequence numbers of the steps in the above-mentioned embodiments does not imply an order of execution. The order of execution of the processes should be determined by their functions and internal logic, and should not constitute any limitation on the implementation of the embodiments of the present disclosure.

### Embodiments 2

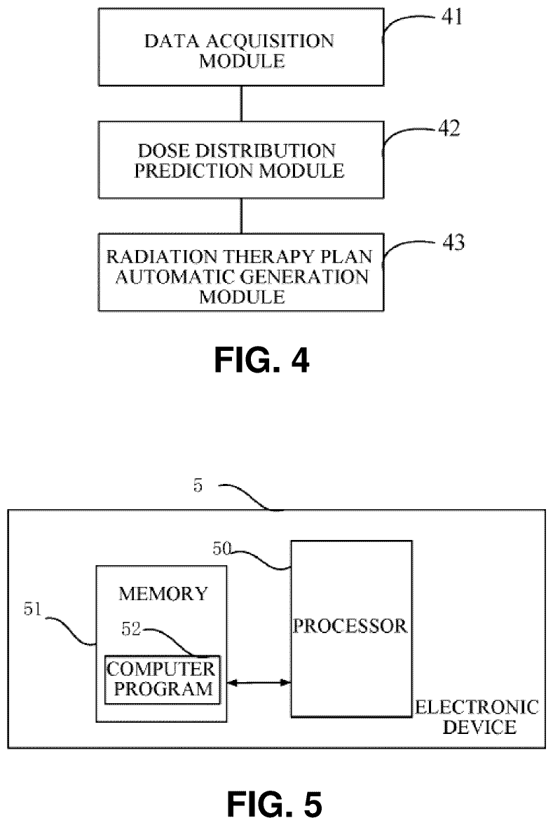

Corresponding to the RT plan generation method described above, FIG. 4 shows a structural block diagram of a function-guided RT plan generation apparatus provided by certain embodiments of the present disclosure. Only parts related to the embodiment of the present disclosure are shown in FIG. 4 for convenience of explanation.

The apparatus includes a data acquisition module 41, a dose distribution prediction module 42, and a RT plan automatic generation module 43.

The data acquisition module 41 is used for acquiring anatomical data and functional data corresponding to a target user. The anatomical data at least models an anatomical structure of a tumor target volume and organ(s) at risk of the target user. The functional data is used to model a lung function distribution of the target user.

The dose distribution prediction module 42 is used for determining the target dose distribution according to the anatomical data and the functional data.

The RT plan automatic generation module 43 is used for determining the function-guided RT plan of the target user based on the target dose distribution.

Since the anatomical data can model the tumor target volume of the target user and the anatomical structure of the organ at risk (that is, the distribution of the tumor target volume and the surrounding organ tissues to be protected), and the functional data can model the actual distribution of the lung function of the target user, it follows that the tumor target volume o of the target user, the organ(s) at risk and the high-low distribution of the lung function can be fully known according to the data. When the target dose distribution is determined, the tissue with good lung function can be avoided as much as possible (that is, the high-function region is avoided, and the dose distribution is concentrated on the tumor region and the low-function region). As a result, the radiation dose to the high-function tissue and the damage to the lung function can be reduced to the greatest extent, and the target dose distribution that is beneficial to the protection of the lung function is obtained. Furthermore, the function-guided RT plan corresponding to the target user is automatically generated according to the target dose distribution. The formulation efficiency, the consistency and the quality of the function-guided RT plan can be effectively improved while the chance of radiation-induced lung injury to the target user is reduced and the effect of RT is improved.

In some embodiments, the dose distribution prediction module 42 includes a prediction unit for taking the anatomical data and the functional data as inputs of a trained prediction model to obtain the target dose distribution output by the prediction model. The prediction model is used to predict the dose distribution matched with the anatomical and functional distributions of the target user according to the anatomical data and the functional data to thereby obtain the target dose distribution.

In some embodiments, the prediction model includes an anatomical encoding network, a function encoding network, a fusion network, and a decoding network. The dose distribution prediction module 42 further includes:
a coding unit for encoding the anatomical data through the anatomical encoding network to obtain anatomical features, and encoding the functional data through the functional encoding network to obtain functional features;
a fusion unit for fusing the anatomical features and the functional features through the fusion network to obtain fusion features; and
a decoding unit for predicting the dose distribution matched with the anatomical and functional distributions according to the fusion features through the decoding network to obtain the target dose distribution.

In some embodiments, the function-guided RT plan generation apparatus further includes:
a sample constructing module for constructing a plurality of training samples, wherein an individual training sample includes anatomical data and functional data corresponding to a user;
a prediction module for taking the individual training sample as inputs of the prediction model to be trained to obtain the predicted dose distribution;
a loss calculation module for determining a loss value according to the predicted dose distribution corresponding to the individual training sample and an actual function-guided planning dose distribution; and
an updating module for updating parameters of the prediction model to be trained according to respective loss values computed for the plurality of training samples to obtain the trained prediction model mentioned above.

In some embodiments, the function-guided RT plan generation apparatus further includes: a function loss calculation module for determining the loss value corresponding to the individual training sample according to the predicted dose distribution, the actual function-guided plan dose distribution, and a functional weight of a target voxel, wherein the functional weight is determined according to the functional data in the individual training sample.

In some embodiments, the RT plan automatic generation module 43 includes: a function-guided dose mimicking unit for generating the function-guided RT plan according to the target dose distribution by a function-guided dose mimicking algorithm adapted to convert the target dose distribution into a RT plan in accordance with an optimization function including at least a target term based on the lung function.

In some embodiments, the RT plan automatic generation module 43 further includes:
a target fluence map calculation unit for solving the optimization function based on the target dose distribution to obtain a target fluence map; and
an optimization unit for converting the target fluence map into machine parameters of a RT machine based on a MLC leaf sequence optimization algorithm and a direct aperture optimization algorithm to obtain the function-guided RT plan.

It should be noted that as the contents such as information interaction and execution process between the above-mentioned apparatus/units are based on the same concept as the method embodiment of the present disclosure, the specific functions and achieved technical effects can be specifically described in the method embodiment section, and are not described in detail here.

### Embodiments 3

FIG. 5 is a schematic diagram of an electronic device provided by certain embodiments of the present disclosure. As shown in FIG. 5, the electronic device 5 includes at least one processor 50 (only one processor is shown in FIG. 5), a memory 51, and a computer program 52 stored in the memory 51 and runnable on the at least one processor 50, which, when executed, implements the steps in any of the various method embodiments mentioned above.

The electronic device 5 can be a computing device such as a desktop computer, a notebook, a PDA, or a cloud server. The electronic device may include, but is not limited to, a processor 50, a memory 51. It will be appreciated by those skilled in the art that FIG. 5 is merely an example of the electronic device 5 and is not intended to limit the electronic device 5, and may include more or fewer components than illustrated, or may combine some components, or different components, for example, may further include input/output devices, network access devices, etc.

The processor 50 may be a central processing unit (CPU) and may further be another general-purpose processor, a digital signal processor (DSP), an application specific integrated circuit (ASIC), a field-programmable gate array (FPGA) or other programmable logic, discrete gate or transistor logic, discrete hardware assemblies, etc. The general-purpose processor may be a microprocessor or the processor may be any conventional processor or the like.

The memory 51 may in some embodiments be an internal storage unit of the electronic device 5, for example a hard disk or memory of the electronic device 5. The memory 51 may in other embodiments also be an external storage device of the electronic device 5, for example a plug-in hard disk, a Smart Media card (SMC), a Secure Digital (SD) card, a Flash Card, etc. provided on the electronic device 5. Furthermore, the memory 51 may further include both an internal storage unit of the electronic device 5 and an external storage device. The memory 51 is used to store an operating system, application programs, BootLoader, data, and other programs, such as program code of the computer program, etc. The memory 51 may further be used to temporarily store data that has been output or is to be output.

It will be apparent to those skilled in the art that, for convenience and brevity of description, only the division of the above-mentioned functional units and modules is illustrated, in actual application, the above-mentioned functions may be assigned by different functional units or modules as needed. That is, the internal structure of the apparatus is divided into different functional units or modules to complete all or part of the functions mentioned above. The various functional units and modules in the embodiments may be integrated in one processing unit, or the various units may exist separately physically, or two or more units may be integrated in one unit, the integrated units may be implemented in the form of hardware or software functional units. In addition, the specific names of each functional unit and module are only for facilitating mutual distinction and are not used for limiting the protection scope of the present disclosure. The specific working processes of the units and modules in the above-mentioned system can refer to the corresponding processes in the above-mentioned method embodiments, and are not described in detail here.

Some embodiments of the present disclosure also provide a network device, which includes: at least one processor, a memory, and a computer program stored in the memory and runnable on the at least one processor. The processor executing the computer program implements the steps in any of the various method embodiments mentioned above.

Embodiments of the present disclosure also provide a computer readable storage medium having stored thereon a computer program that, when executed by a processor, performs the steps as set forth in various method embodiments mentioned above.

Embodiments of the present disclosure further provide a computer program product that, when run on an electronic device, causes the electronic device to perform steps as set forth in various method embodiments mentioned above.

The integrated units, if implemented in the form of software functional units and sold or used as stand-alone products, may be stored in a computer-readable storage medium. Based on this understanding, the present disclosure implements all or part of the processes in the methods of the embodiments mentioned above, which may be performed by computer programs that direct associated hardware, which may be stored in a computer readable storage medium that, when executed by a processor, implements the steps of the various method embodiments mentioned above. The computer program includes computer program code which may be in the form of source code, object code, executable files or some intermediate form, etc. The computer readable medium may include at least any entity or apparatus capable of carrying computer program code to a photographing apparatus/electronic device, a recording medium, a computer memory, a read-only memory (ROM), a random access memory (RAM), an electrical carrier wave signal, a telecommunication signal, and a software distribution medium. For example, a USB flash drive, a removable hard disk, a magnetic disk or optical disk, etc. In some jurisdictions, computer readable media may not be electrical carrier signals and telecommunications signals in accordance with legislative and patent practice.

In the above-mentioned embodiments, the description of each embodiment has its own focus, and for parts not detailed or recorded in one embodiment, reference can be made to the related description of other embodiments.

Those skilled in the art will appreciate that the units and algorithm steps of each example described in conjunction with the embodiments disclosed herein can be implemented by electronic hardware, or a combination of computer software and electronic hardware. Whether these functions are performed in hardware or software depends on the specific application and design constraints of the technical solution. Professional technicians can use different methods to implement the described functions for each specific application, but such implementation should not be considered to be beyond the scope of this application.

In the embodiments provided in this application, it should be understood that the disclosed apparatus/network equipment and method may be implemented in other ways. For example, the embodiments of the apparatus/network device mentioned above are merely illustrative, for example, the division of the module or unit is merely a logical functional division, and there may be other ways of division in actual implementation, for example, multiple units or assemblies may be combined or integrated into another system, or some features may be omitted or not performed. On the other hand, the coupling or direct coupling or communication connection shown or discussed with respect to each other may be an indirect coupling or communication connection through some interface, apparatus, or unit, which may be in an electrical, mechanical, or other form.

The units described as separate components may or may not be physically separated, and the components shown as units may or may not be physical units, i.e. may be located in one place, or may be distributed over multiple network units. Some or all of the units may be selected according to actual needs to achieve the purpose of the present embodiment.

The above-described embodiments are only for illustrating the technical scheme of the present disclosure, and are not meant to be limiting; although the present disclosure has been described in detail with reference to the foregoing embodiments, it will be understood by those skilled in the art that modifications may be made to the technical solutions described in the foregoing embodiments, or equivalents may be substituted for some of the technical features thereof, and these modifications or substitutions do not depart from the spirit and scope of the corresponding technical solutions in the embodiments of the present disclosure, and are intended to be included within the scope of the present disclosure.

What is claimed is:

1. A function-guided radiation therapy (RT) plan generation method, the method comprising:
acquiring anatomical data and functional data corresponding to a target user, wherein the anatomical data at least models an anatomical structure of a tumor target volume and organ(s) at risk of a target user, and wherein the functional data is used to model a distribution condition of a lung function of the target user;
determining a target dose distribution according to the anatomical data and the functional data; and
determining a function-guided RT plan for the target user based on the target dose distribution.

2. The method of claim 1, wherein the determining of the target dose distribution based on the anatomical data and the functional data includes:
using a prediction model to obtain the target dose distribution after the prediction model is trained, wherein the prediction model is designed to establish a relationship between the anatomical and functional data and corresponding dose distribution, thereby obtaining the target dose distribution according to the anatomical and functional data of the target user.

3. The function-guided RT plan generation method of claim 2, wherein the prediction model includes an anatomical encoding network, a function encoding network, a fusion network, and a decoding network, and wherein the using of the trained prediction model to obtain the target dose distribution includes:
encoding the anatomical data through the anatomical encoding network to obtain anatomical features, and encoding the functional data through the functional encoding network to obtain functional features;
fusing the anatomical features and the functional features through the fusion network to obtain fusion features; and
predicting the dose distribution matched with the anatomical and functional distributions according to the fusion features through the decoding network to obtain the target dose distribution.

4. The function-guided RT plan generation method of claim 2, wherein the prediction model is trained by the following steps:
constructing a plurality of training samples, wherein an individual training sample includes anatomical data and functional data corresponding to a user;
taking the individual training sample as inputs of the prediction model to be trained to obtain the predicted dose distribution;
determining a loss value according to the predicted dose distribution corresponding to the individual training sample and an actual function-guided planning dose distribution; and
updating parameters of the prediction model to be trained according to respective loss values computed for the plurality of training samples to obtain the prediction model as trained.

5. The function-guided RT plan generation method of claim 4, wherein the determining of the loss value according to the predicted dose distribution corresponding to the individual training sample and the actual function-guided planning dose distribution includes:
determining the loss value corresponding to the individual training sample according to the predicted dose distribution, the actual function-guided plan dose distribution, and a functional weight of a target voxel, wherein the functional weight is determined according to the functional data in the individual training sample.

6. The function-guided RT plan generation method according to claim 1, wherein the determining of the function-guided RT plan of the target user based on the target dose distribution includes:
generating the function-guided RT plan according to the target dose distribution by a function-guided dose mimicking algorithm adapted to convert the target dose distribution into a RT plan in accordance with an optimization function including at least a target term based on the lung function.

7. The function-guided RT plan generation method of claim 6, wherein the generating of the function-guided RT plan according to the target dose distribution by the function-guided dose mimicking algorithm includes:
solving the optimization function based on the target dose distribution to obtain a target fluence map; and
converting the target fluence map into machine parameters of a RT machine based on a MLC leaf sequence optimization algorithm and a direct aperture optimization algorithm to obtain the function-guided RT plan.

8. A function-guided radiation therapy (RT) plan generation apparatus comprising:
a data acquisition module for acquiring anatomical data and functional data corresponding to a target user, wherein the anatomical data at least models an anatomical structure of a tumor target volume and organ(s) at risk of a target user, and the functional data is used to model a distribution condition of a lung function of the target user;
a dose distribution prediction module for determining a target dose distribution according to the anatomical data and the functional data; and
an automatic RT plan generation module for determining a function-guided RT plan for the target user based on the target dose distribution.

9. An electronic device including a memory, a processor, and a computer program stored in the memory and runnable on the processor, wherein the processor, when executing the computer program, implements the method according to claim 1.

10. A computer program product, wherein when the computer program product is run on an electronic device, the electronic device is caused to execute the method according to claim 1.
